(19) United States
(12) Patent Application Publication
(10) Pub. No.: US 2020/0113742 A1
(43) Pub. Date: Apr. 16, 2020

(54) Textile Orthosis with Asymmetrical Edge

(71) Applicant: LABORATOIRES INNOTHERA, Arcueil (FR)

(72) Inventor: Isabelle FOUCHER, Moriville (FR)

(73) Assignee: LABORATOIRES INNOTHERA, Arcueil (FR)

(21) Appl. No.: 16/599,611

(22) Filed: Oct. 11, 2019

(30) Foreign Application Priority Data
Oct. 12, 2018 (FR) 18 59462

(51) Int. Cl. A61F 13/08; D04B 1/26

(52) U.S. Cl. CPC A61F 13/08; D04B 1/265

(57) ABSTRACT

An orthosis chosen from an elastic venous retention orthosis and an elastic venous compression orthosis is intended to treat a venous disorder of a lower limb of a patient in a service position in which it is fitted, via an opening, on the lower member, the orthosis having a ribbed top and a leg part, the ribbed top including a rim that delimits the opening, the orthosis being characterized in that the front part of the rim of the ribbed top has, on average, an altitude greater than that of the rear part of the rim of the ribbed top.

### TECHNICAL FIELD

The present invention relates to an elastic venous compression or retention orthosis (or EVC orthosis, or “orthosis”) which, in the service position, extends substantially as far as the knee of the lower limb of a patient on which it is fitted.

### PRIOR ART

An EVC orthosis of this kind, formerly known by the term “retention stocking (or hose)” or “retention tights”, is a textile medical device producing a therapeutic effect through retention and/or compression of the lower limbs, as opposed to “support stockings” (or even “support hoses” or “anti-fatigue stockings”) and “fashion stockings”, which are not medical devices with a therapeutic purpose.

It is intended to treat a venous disorder of a patient.

The pressure profile exerted by an orthosis usually decreases upward from the ankle.

Depending on the type of orthosis, the pressure measured at the ankle can in particular vary from 10 to over 36 mmHg (i.e. 13 to 48 hPa, although mmHg is commonly used as the unit of measurement of pressure in the field of phlebology and of medical compression).

The orthoses are divided into four textile classes according to the ASQUAL system, from class I (13 to 20 hPa≈10 to 15 mmHg at the ankle) to class IV (>48 hPa≈>36 mmHg at the ankle).

The upper part of the orthosis, which defines the opening via which it is fitted, traditionally has a ribbed top. A ribbed top is a band of knitting which is extensible in the circumferential direction and which, once the orthosis has been fitted on the limb, has the function of locally exerting a textile pressure for holding the orthosis in position.

In a seated position, when the leg is bent, it very often happens that the skin behind the knee forms folds. The greater the level of compression supplied by the product, the more this phenomenon can prove uncomfortable, or even unbearable, making compliance with treatment difficult.

An aim of the invention is to make available an orthosis that at least partially overcomes this problem.

### SUMMARY OF THE INVENTION

The invention proposes an elastic venous compression or retention orthosis having a ribbed top and a leg part, the ribbed top comprising a rim that delimits an opening via which the orthosis, in a service position, is fitted on a lower limb of a patient, said orthosis extending, in the service position, substantially as far as the knee of said lower limb.

The orthosis is characterized in that the front part of the rim of the ribbed top has, on average, an altitude greater than that of the rear part of the rim of the ribbed top, preferably greater by more than 0.2%, 0.5%, 1%, 2%, 3%, 4% or 5% than the altitude of the rear part.

Advantageously, this asymmetry limits the interactions between the orthosis and the folds of the skin behind the knee when the patient bends his or her leg, and this enhances comfort and improves treatment compliance.

Preferably, the amplitude of the variation in altitude along the rim of the ribbed top is more than 0.5 cm, preferably more than 0.7 cm, preferably more than 0.8 cm, preferably more than 0.9 cm, and/or preferably less than 3.0 cm, preferably less than 2.0 cm, preferably less than 1.5 cm, preferably less than 1.2 cm.

Preferably, the orthosis belongs to class I, II, III or IV according to the ASQUAL system.

An orthosis according to the invention can in particular also have one or more of the following optional and preferred features:
over 80%, preferably over 90%, preferably 100% of the length of the front part of the rim is at an altitude above the highest point of the rear part of the rim;
over 80%, preferably over 90%, preferably 100% of the length of the rear part of the rim is at an altitude below the lowest point of the front part of the rim;
the inclination of the rim, that is to say the variation in altitude per unit length of the rim, is constant at every point of the rim from the lowest point to the highest point of the rim;
the altitudes of the front and rear ends of the rim are maximal and minimal, respectively;
the whole of the ribbed top is knitted in continuity with the leg part;
the ribbed top has a stitch height that varies depending on the local altitude of the rim.

### BRIEF DESCRIPTION OF THE FIGURES

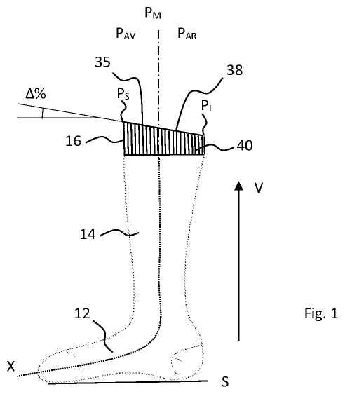

Further features and advantages of the invention will become clear from reading the following detailed description and from studying the attached drawing in which FIG. 1 shows a schematic side view of an orthosis having a ribbed top whose rim is of variable altitude.

This FIGURE depicts the median transverse plane P_{M} passing along the main direction X of the orthosis and separating the space, and in particular the ribbed top, into a front part P_{AV} and a rear part P_{AR}. A “transverse” plane is a plane parallel to the general plane of the patient's body in the service position.

### Definitions

The “service position” of an orthosis corresponds to a position of use, that is to say a position in which it is fitted on a lower limb of a patient who is standing up.
The “at rest” or “flat” position of an orthosis is a position in which the orthosis is not being worn, for example before the first use, and extends, seen from the side, in the same way as in the service position, as in FIG. 1.
The pressure exerted at a point by an orthosis is, according to Laplace's law, a pressure referred to as a “textile pressure” within the meaning of French standard NF G30-102b, inversely proportional to the radius of curvature at this point.
The “altitude” corresponds to a level in the vertical direction V (shown in FIG. 1) in the service position. It is measured from the lower end of the orthosis in the rest position, that is to say from the plane S in FIG. 1. This plane represents the ground in the service position. A “height” is a difference in altitude. The “inclination” of the rim is the altitude gradient.
The dimensions, in particular the variation in altitude of the rim, are traditionally measured on a Hohenstein leg model in order to simulate the service position.
A “stitch” is a way of interlacing the knit and weft threads.

Unless indicated to the contrary, the dimensions, such as the height, the variations in altitude, such as the inclination, and the positions, such as “front”, “rear”, “upper” or “lower”, are given with reference to the at rest position, the orthosis being arranged vertically as shown in FIG. 1.

Unless indicated to the contrary, “having”, “comprising”, “including”, or variations thereof, correspond to non-exclusive inclusion.

### DETAILED DESCRIPTION

Reference sign 10 designates in a general manner an orthosis according to the invention, extending along the main direction X.

The orthosis 10, of overall anatomical shape, comprises
a foot part 12 which envelops the foot, extending from the toes as far as the malleoli, covering the instep,
a leg part 14 which is extensible in a longitudinal direction (that is to say in the main direction X) and in the circumferential direction (radial extensibility), extending from the malleolar region, enveloping the ankle and the calf, up to a level situated below the knee; and
a ribbed top 16 extensible mainly in the circumferential direction, typically a knitted part, which continues the leg part 14.

An orthosis according to the invention, as shown in FIG. 1, takes the form of a knee-high sock, referred to as “knee-length” or “half-stocking” in the “AD” format, meaning that the ribbed top reaches to just below the knee when the orthosis is in the service position. Outwardly, it therefore has the same appearance as traditional “fashion” socks, but differs from these in terms of the choice of thead and a knit that allows effective therapeutic compression to be obtained, preferably compression of class II.

The orthosis 10 can also be without a foot part 12 (stocking or hose of the “open foot” type).

The orthosis 10 is conventionally made from a knitted stitch and, in the leg part, incorporates an elastic weft thread, generally made of a covered elastane.

Under the effect of the application of the orthosis to the patient's limb, the elastic weft thread of the leg part generates a return force which tends to cause the orthosis to slip toward the foot.

The holding pressure exerted by the ribbed top therefore needs to be particularly high, and the possible folds are particularly uncomfortable, or even unbearable.

In the service position, the ribbed top 16 forms a substantially cylindrical belt, preferably of variable height (measured along the direction V).

The maximum height of the ribbed top 16 is preferably more than 2.0 cm, preferably more than 3.0 cm, preferably more than 4.0 cm, and/or preferably less than 8 cm, preferably less than 6 cm, preferably less than 5 cm.

The minimum height of the ribbed top 16 is preferably more than 1.0 cm, preferably more than 2.0 cm, preferably more than 3.0 cm, and/or preferably less than 8 cm, preferably less than 6 cm, preferably less than 5 cm, preferably less than 4.0 cm.

The opening 38 of the orthosis is delimited by a rim 35, defined by the ribbed top.

According to the invention, the altitude of the rim 35 is variable, the amplitude of the variation along the rim 35, that is to say the difference between the altitude of the one or more upper points P_{S} of the rim 35 (that is to say having the highest altitude) and the altitude of the one or more lower points P_{I} of the rim 35 (that is to say having the lowest altitude), preferably being more than 0.5 cm, preferably more than 0.7 cm, preferably more than 0.8 cm, preferably more than 0.9 cm, and/or preferably less than 3.0 cm, preferably less than 2.0 cm, preferably less than 1.5 cm, preferably less than 1.2 cm.

This configuration advantageously makes it possible to reduce the presence of folds in the ribbed top.

In one embodiment, the variation in the altitude of the rim 35, in absolute value, that is to say the “inclination” Δ % of the rim 35, is substantially constant when the orthosis is looked at in the flat state, seen from the side, as is shown. Preferably, the ratio of the difference between the maximum inclination (greatest) and the minimal elevation (weakest), divided by the maximum inclination, is less than 10%, preferably less than 5%, preferably substantially zero.

In one embodiment, the rim 35 is inclined over more than 50%, more than 70%, more than 90%, or even substantially over 100% of its length.

Preferably, the rim 35 has a constant inclination over more than 50%, more than 70%, more than 90%, or even substantially over 100% of its length.

Preferably, the front part P_{AV} of the ribbed top has, on average, an altitude greater than that of the rear part P_{AR}.

Preferably, the one or more upper points are all arranged in the front part P_{AV} of the ribbed top. Preferably, the one or more lower points are all arranged in the rear part P_{AR} of the ribbed top.

The terms “part AVx %” and “part ARx %” designate the parts of the rim 35 of which the length represents “x %” of the total length of said rim and which extend farthest to the front and farthest to the rear, respectively, of the ribbed top. The front part P_{AV} of the ribbed top is therefore the part AV50%.

Preferably, the one or more upper points P_{S} are all arranged in the part AV30%, preferably in the part AV20%, preferably in the part AV10%, preferably in the part AV5%. Preferably, the one or more lower points are all arranged in the part AR30%, preferably in the part AR20%, preferably in the part AR10%, preferably in the part AR5%. In a preferred embodiment, as in FIG. 1, the rim has a single upper point P_{S} and a single lower point P_{I}, which are arranged at the front and rear ends of the rim, respectively.

Preferably, the ribbed top has a uniform appearance.

The ribbed top 16 preferably has ribs 40 extending preferably substantially vertically. The ribs 40 preferably extend over the entire contour of the ribbed top, as is shown. The ribs 40 form pleats that can easily be stretched out. Advantageously, the ribs 40 also make it possible to reduce substantially the tendency of the knit to roll up on itself or form wrinkles.

The ribbed top 16 can, for example, have a jersey stitch with a ribbed pattern, for example a 1×1 jersey rib pattern (that is to say with 1 wale raised×1 wale recessed, corresponding to a knit with 1 stitch in place 1, ×1 purl stitch).

The weft thread used for the leg part can be identical to or different than that used for the ribbed top.

In one embodiment, the weft threads of the leg part and of the ribbed top are elastic. They preferably have elastane and/or an elasto-diene.

Preferably, the same stitch thread is used for the leg part and for the ribbed top.

Preferably, the linear density of the core of the stitch thread of the ribbed top is below 44 dTex, preferably below 30 dTex, preferably below 25 dTex, preferably below 20 dTex, and/or above 10 dTex, preferably above 15 dTex.

Preferably, the linear density of the covering thread of the stitch thread of the ribbed top is below 60 dTex, preferably below 50 dTex, preferably below 45 dTex, and/or above 20 dTex, preferably above 30 dTex, preferably above 40 dTex.

Preferably, the weft thread of the ribbed top is a covered thread, preferably a double-covered thread.

Preferably, the linear density of the core of the weft thread of the ribbed top is below 150 dTex, preferably below 140 dTex, preferably below 135 dTex, and/or above 100 dTex, preferably above 110 dTex, preferably above 120 dTex, preferably above 125 dTex.

Preferably, the linear density of the covering thread of the weft thread of the ribbed top is below 40 dTex, preferably below 30 dTex, preferably below 25 dTex, and/or above 10 dTex, preferably above 15 dTex, preferably above 20 dTex.

Preferably, the ribbed top is knitted in continuity with the leg part 14. In other words, it is not a part that is added on, but a part that is knitted in the same sequence on the knitting machine. At the crossing of the boundary between the leg part 14 and the ribbed top 16, there is therefore no change in the weft thread, nor is there any change in the stitch thread.

Preferably, this boundary is substantially horizontal, as is shown in FIG. 1.

In one embodiment, the only difference between the leg part and the ribbed top lies in the stitch.

In a preferred embodiment, the ribbed top has a stitch height that varies depending on the local altitude of the rim. Preferably, the stitch height of the part of the rim extending below a defined part of the rim increases the greater the altitude of this part of the rim.

For example, in the embodiment in FIG. 1, the stitch height can be greater on the front part of the rim than on the rear part of the rim.

These differences in stitch height can be obtained by the use of clamping cams on a circular knitting machine.

An orthosis according to the invention has proven particularly effective in limiting the interactions between the orthosis and the folds of the skin immediately below the back of the knee. This results in improved treatment compliance.

Of course, the invention is not limited to the embodiments described and shown, which have been provided only for illustrative purposes.

1. Orthosis chosen from an elastic venous retention orthosis and an elastic venous compression orthosis, said orthosis being intended to treat a venous disorder of a lower limb of a patient in a service position in which it is fitted, via an opening, on said lower limb, said orthosis having a ribbed top and a leg part, the ribbed top comprising a rim that delimits said opening, said orthosis extending, in the service position, substantially as far as the knee of said lower limb,
wherein the front part of the rim of the ribbed top has, on average, an altitude greater than that of the rear part of the rim of the ribbed top.

2. Orthosis according to claim 1, in which the amplitude of the variation in altitude along the rim is greater than 0.8 cm.

3. Orthosis according to claim 1, in which over 80% of the length of the front part of the rim is at an altitude above the highest point of the rear part of the rim, and/or in which over 80% of the length of the rear part of the rim is at an altitude below the lowest point of the front part of the rim.

4. Orthosis according to claim 1, in which the inclination of the rim is constant at every point of the rim from the lowest point to the highest point of the rim.

5. Orthosis according to claim 1, in which the altitudes of the front and rear ends of the rim are maximal and minimal, respectively.

6. Orthosis according to any one of the preceding claim 1, in which the whole of the ribbed top is knitted in continuity with the leg part.

7. Orthosis according to claim 1, in which the ribbed top has a stitch height that varies depending on the local altitude of the rim.

8. Orthosis according to claim 1, of class I, II, III or IV according to the ASQUAL system.
